(19) United States
(12) Patent Application Publication
(10) Pub. No.: US 2014/0016090 A1
(43) Pub. Date: Jan. 16, 2014

(54) DEVICE AND PROCESS FOR DETERMINING THE DOMINANT EYE OF A PATIENT

(71) Applicants: Thierry Bonnin, Charenton Le Pont (FR); Fabien Divo, Charenton Le Pont (FR); Cecile Petignaud, Charenton Le Pont (FR); Isabelle Poulain, Charenton Le Pont (FR); Philippe Pinault, Charenton Le Pont (FR); Laurent Roussel, Charenton Le Pont (FR)

(72) Inventors: Thierry Bonnin, Charenton Le Pont (FR); Fabien Divo, Charenton Le Pont (FR); Cecile Petignaud, Charenton Le Pont (FR); Isabelle Poulain, Charenton Le Pont (FR); Philippe Pinault, Charenton Le Pont (FR); Laurent Roussel, Charenton Le Pont (FR)

(21) Appl. No.: 14/006,970

(22) PCT Filed: Mar. 8, 2012
(86) PCT No.: PCT/FR2012/000083

§ 371 Date: Sep. 24, 2013

(30) Foreign Application Priority Data
Mar. 25, 2011 (FR) 1100896

(51) Int. Cl. A61B 3/10

(52) U.S. Cl. CPC A61B 3/10
USPC 351/204; 351/246

(57) ABSTRACT

A device and process for determining the dominant eye of a patient (10), wherein the device includes a target (120), a camera (110) for acquiring an image of the patient's face, and a mask (200) to be positioned in front of the patient's face. The mask includes; a closure face (210) suitable for hiding the target from the patient while enabling the camera to see the patient's face, and a viewing window (220) for the patient to view the target and which is located through the closure face in order to enable the patient to view the target with only one of his two eyes.

### TECHNICAL FIELD TO WHICH THE INVENTION RELATES

The present invention in general relates to the field of eyewear.

It more particularly relates to a measuring device and method for acquiring the dominant eye of a patient.

### TECHNOLOGICAL BACKGROUND

The manufacture of a pair of spectacles is split into six main operations:
acquiring patient related parameters;
calculating the shapes of the optical faces of the lenses, depending on these acquired parameters;
molding and machining of the optical faces of the lenses;
acquiring data relating to the spectacle frame selected by the patient, including, in particular, the shapes of the outlines of the rims of this frame;
centering the ophthalmic lenses, which consists in suitably positioning the outlines of the rims on each lens so that, once they have been machined to the shape of these outlines and then mounted in the frame, these lenses fulfill, as well as can be expected, the optical functions for which they were designed; and
shaping the lenses.

Currently, in order to improve the visual comfort of patients, there are researches to optimize the optical shapes and performance of lenses, especially those of lenses exhibiting a progressive power variation (commonly called “progressive lenses”), and to improve how well they are centered in the rims of the spectacle frame.

To do this, an increasing number of patient related parameters must be taken into consideration.

Among these parameters, it is now sought to determine the dominant eye (or “master eye”) of the patient, especially in order to personalize the calculation and machining of the lenses of the patient.

Various empirical methods are known for determining the dominant eye of the patient, which, in practice, prove to be unreliable since they are based entirely on the skill and ease with which the patient can implement them.

One very common method is the “hole-in-card” method also called the “hole-in-the-card test” or the Dolman method.

This method proves to be one of the surest ways of identifying the dominant eye of an individual. It consists in:
giving the patient a card with a hole in its center;
asking the patient to hold this card in both hands, with straight arms; and then in
asking the patient to keep both eyes open and to sight a target, located at a distance in front of them, through the hole (in the sighting position the subject perceives the target centered in the hole).

The patient then closes each of their two eyes in alternation in order to identify their dominant eye, which, in practice, is the eye aligned with the target and the hole. Thus, if the target is still centered in the hole when the patient shuts their left eye, their right eye is dominant. Conversely, if the target is still centered in the hole when they shut their right eye, their left eye is dominant.

The identification of the dominant eye using this method is therefore subjective since it depends on verbal feedback from the patient regarding their perception of the target.

### SUMMARY OF THE INVENTION

In order to remedy the inadequacy of a result based on a subjective response from the patient, the present invention provides an objective measuring device and a method allowing the dominant eye of the patient to be determined, which is at least partially automated in order to reduce the risk of error resulting from a subjective measurement.

More particularly, according to the invention, a device is provided for determining the dominant eye of a patient, which comprises:
a target visible by the patient in the measuring position;
a means for acquiring an image of the face of the patient in the measuring position; and
a mask to be positioned between, on the one hand, the face of the patient in the measuring position, and on the other hand, the target and the acquisition means, comprising:
   a) an obstructing panel:
      a1) that has dimensions such that it is capable of being interposed between the target and the two eyes of the patient in the measuring position;
      a2) that transmits, at least momentarily, only at most some of the light propagating from the target toward the patient in order to hide the target from the patient in the measuring position; and
      a3) that transmits, at least momentarily, at least some of the light propagating from the patient toward the acquisition means in order to allow the acquisition means to acquire an image of the face of the patient in the measuring position; and
   b) a sighting window used by the patient to sight the target:
      b1) that is located in the obstructing panel;
      b2) that transmits the light propagating from the target toward the patient, in order to allow the patient to sight the target; and
      b3) that has dimensions such that the target is visible only by a single eye of the patient in the measuring position.

According to the invention, a method for determining the dominant eye of a patient is also provided, which comprises:
a step in which the patient and the mask are positioned in the field of the means for acquiring images, the mask in front of the two eyes of the patient;
a step in which the patient sights the target through the sighting window;
a step in which the acquisition means acquires an image of the patient sighting the target, in which image the face of the patient and the mask appear; and
a step in which the dominant eye of the patient is deduced depending on the position of the mask relative to the face of the patient in the acquired image.

The principle used here to determine the dominant eye of the patient consists in allowing the patient to sight the target naturally through the sighting window in the mask, using either one of their two eyes, while preferably keeping both of their eyes open. The eye naturally used to sight the target is in practice the dominant eye of the patient.

The automation of the method then consists in acquiring an image of the patient while they are sighting this target.

Specifically, once the target is being correctly sighted by the patient, and while at least one of their two eyes is still hidden behind the obstructing panel of the mask, the acquisition means is able, because of the light transmission properties of the obstructing panel, to acquire an image in which the mask and both eyes of the patient appear.

By establishing in this image the position of the sighting window in the mask relative to both eyes of the patient, it is thus possible to deduce therefrom which of the two eyes of the patient is their dominant eye.

Since this deduction step is not carried out by the patient themselves, it is observed that the latter adopts a more natural behavior when sighting the target and may perform the sighting task while keeping both eyes constantly open, ideal condition for ensuring there is real competition between both eyes of the patient.

The following are other advantageous and nonlimiting features of the device according to the invention:
the obstructing panel and the window are passive in the sense that they have light transmission properties that are invariable in time;
the obstructing panel polarizes light linearly or circularly, and a pair of spectacles or equivalent is provided with which the patient is to be equipped in the measuring position, which comprises lenses that are polarized linearly or circularly, respectively, the lenses and the obstructing panel being rectilinearly polarized in orthogonal directions or circularly polarized in opposite senses;
the obstructing panel is active in the sense that it has variable light transmission properties;
the obstructing panel and the sighting window together form a liquid-crystal screen;
the sighting window is inactive in the sense that it has invariable light transmission properties, and the obstructing panel comprises an activatable layer controlled to exhibit complete opacity or transparency;
a display screen is provided, said display screen being capable of displaying an image of the patient in the measuring position, said image being acquired by the acquisition means;
an information processing unit is provided, said information processing unit being capable of processing an image of the patient in the measuring position and of deducing therefrom the dominant eye of the patient, said image being acquired by the acquisition means; and
the mask comprises a chassis equipped with at least one maneuvering handle, and is portable by the patient.

The following are other advantageous and nonlimiting features of the method according to the invention:
the deduction step consists in displaying the acquired image on a display screen;
in the deduction step, the acquired image is processed in order to calculate, on the one hand, the positions of the two eyes of the patient, and on the other hand, the position of the sighting window relative to the two eyes of the patient;
the patient being equipped with a pair of spectacles, in the deduction step, the acquired image is processed in order to calculate, on the one hand, the positions of the two rims of the pair of spectacles, and on the other hand, the position of the sighting window relative to the two rims;
the mask comprising a visible reference mark, the acquired image is processed in order to calculate the position of the face of the patient relative to the reference mark;
the image acquisition is manually controlled;
the image acquisition is carried out continuously or at regular intervals, and, in the deduction step, provision is made for one of said acquired images to be selected;
the image is selected when the position of the face of the patient relative to the mask has stabilized; and
a step of displaying the acquired image is provided.

### DETAILED DESCRIPTION OF AN EMBODIMENT

The following description, which refers to the appended drawings, given by way of nonlimiting example, will allow the subject matter of the invention and how it can be implemented to be better understood.

In the appended drawings:

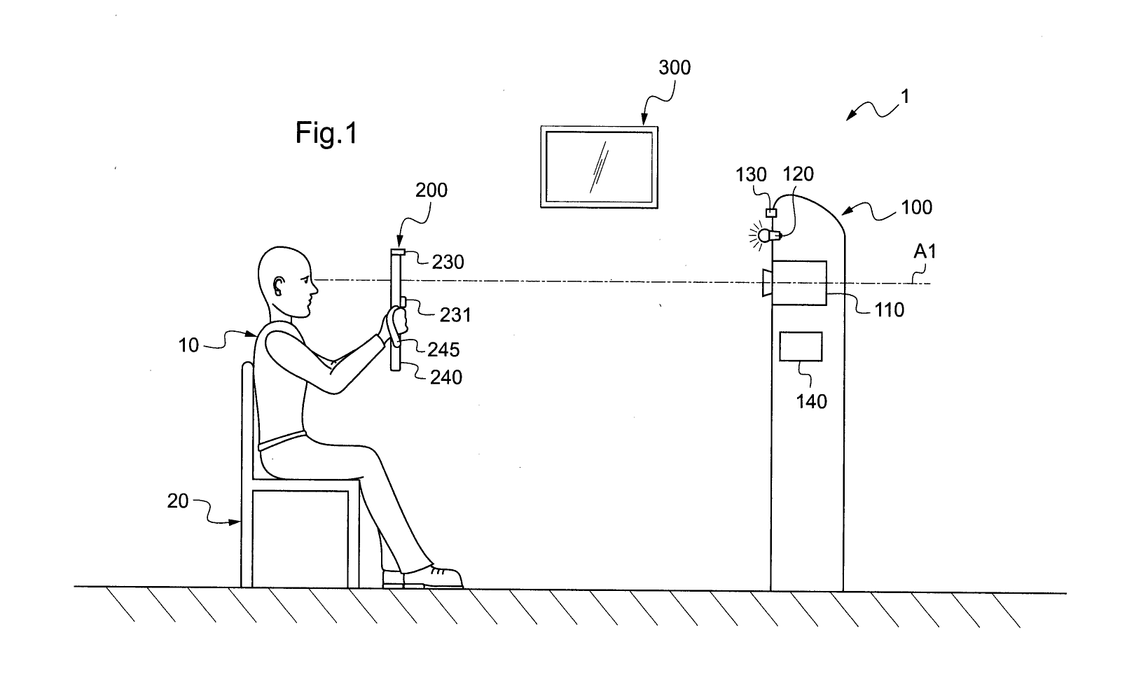

FIG. 1 is a schematic side view of a device for determining the dominant eye of a patient according to the invention;

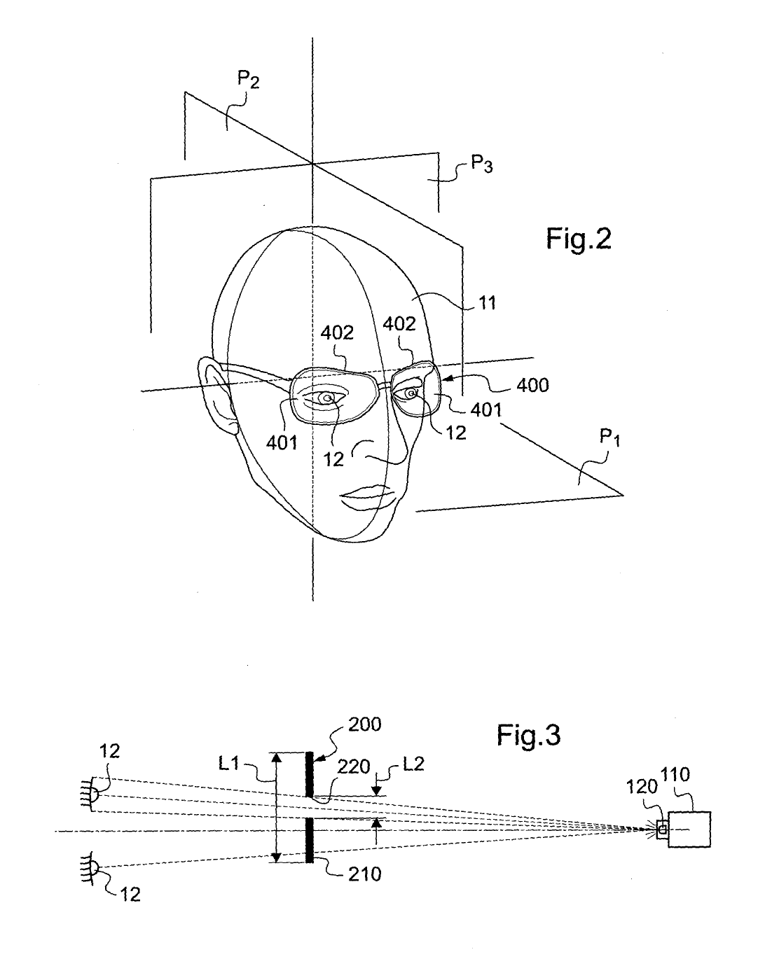

FIG. 2 is a schematic view of the face of the patient;

FIG. 3 is a schematic top view of the device in FIG. 1; and

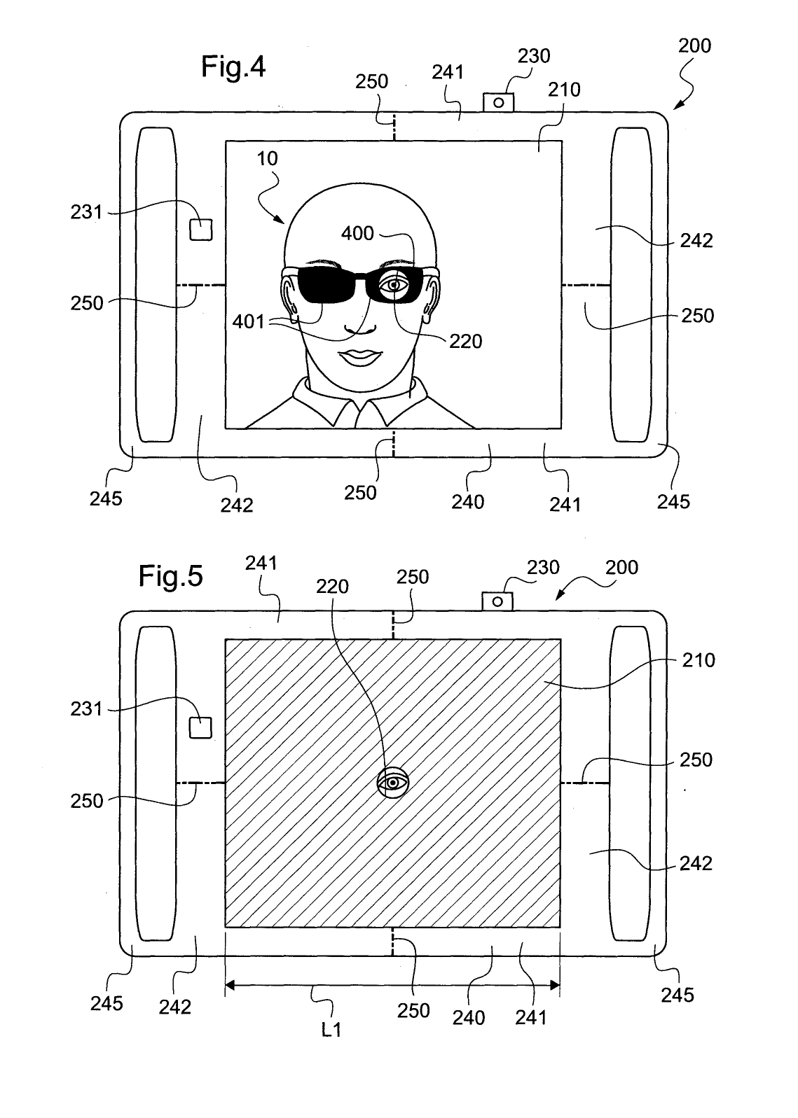

FIGS. 4 and 5 are schematic views of two variant embodiments of the mask of the device in FIG. 1.

To start with, it will be noted that identical or similar elements in the various variant embodiments of the invention shown in the various figures will, as far as possible, be referenced by the same reference symbols and will not be described each time.

FIG. 1 shows a device 1 for determining the dominant eye of a patient 10.

This determining device 1 comprises three main elements, namely a target 120 visible by the patient 10, a means 110 for acquiring an image of the face of the patient 10, and a mask 200 to be interposed between, on the one hand, the face of the patient 10, and on the other hand, the target 120 and the acquisition means 110.

It will be noted at this point that the target 120 and the acquisition means 110 could be merged in the sense that the acquisition means, visible by the patient, could form the target to be sighted.

Specifically, the target 120 that the patient 10 must sight may consist of an object, a pattern, a light or more generally any means capable of being seen by the patient 10.

Such as shown in FIG. 1, the target 120 consists of a light source, in this case a light-emitting diode.

This target 120 is here located on a column, called a totem 100, placed facing the seat 20 on which the patient 10 sits during the measurement.

This target 120 is more precisely located substantially at the same height as the eyes 12 of the patient 10, and at a distance from the latter of between 1.2 and 1.5 meters.

The acquisition means 110 is here also located on the totem 100, substantially at the same height as the eyes 12 of the patient 10.

Here it is located under the target 120 and near the latter.

This acquisition means 110 may consist of any system able to acquire an image of the patient 10.

Such as shown in FIG. 1, this acquisition means is a digital camcorder 110 of optical axis A1. It could also be formed by a digital camera.

As is better shown in FIG. 5, the mask 200 comprises at least one obstructing panel 210 and a sighting window 220 located in the obstructing panel 210.

As FIG. 3 shows, the sighting window 220 is designed to allow the patient 10 to sight the target 120 using only one of their two eyes 12. It will be noted here that the eye that is most naturally used to sight the target is in practice the dominant eye of the patient 10.

The obstructing panel 210 of the mask 200 for its part is designed to allow the target 120 to be hidden from the other eye of the patient 10. It is moreover advantageously designed in such a way that it allows the acquisition means 110 to acquire an image of at least part of the face of the patient 10 while they are sighting the target 120 (this part of the face being enough to deduce from the acquired image which of the two eyes 12 of the patient is their dominant eye).

In this case, the mask 200 also comprises a chassis 240 taking the form of a frame that encircles the obstructing panel 210 and that allows the patient to maneuver the mask 200 more easily.

As FIG. 5 shows, the obstructing panel 210 of the mask 200 has dimensions such that it is capable of being interposed between the target 120 and the two eyes 12 of the patient 10.

It is in particular capable of hiding the target 120 from one of the eyes 12 of the patient 10 while the patient is sighting the target 120 with the other eye through the sighting window 220.

In the example shown in FIGS. 3 and 5, the obstructing panel has a rectangular shape with a width L1 of between 100 and 300 millimeters and a height of between 100 and 300 millimeters.

In order to hide the target 120 from at least one of the two eyes 12 of the patient 10, provision is made for the obstructing panel 210 to transmit, at least momentarily, only at most some of the light propagating from the target 120 toward the patient 10.

In order to allow the camcorder 110 to acquire an image of the face of the patient 10, provision is made for the obstructing panel 210 to transmit, at least momentarily, at least some of the light propagating from the face of the patient 10 toward the camcorder 110.

Detailed embodiments of this obstructing panel 210 will be given in the rest of this text.

The sighting window 220, which is provided in order to allow the patient 10 to sight the target 120, is located a distance away from the edges of the obstructing panel 210 in order to ensure that, while the patient 10 is sighting the target 120 with one eye, their other eye remains hidden behind the obstructing panel 210 of the mask 200.

The sighting window 220 is preferably located at mid-width in the obstructing panel 210.

It has dimensions such that the target 120 is visible only by just one of the two eyes 12 of the patient 10 when the patient 10 holds the mask 200 at arm's length (FIG. 1).

In the example shown in the figures, the sighting window 220 takes the form of a disk centered on the middle of the obstructing panel 210, with a diameter L2 between 10 and 40 millimeters and preferably equal to 25 millimeters.

The chassis 240, which takes the form of a frame, for its part comprises two longitudinal arms 241 and two lateral arms 242. Its two lateral arms 242 have substantial widths and are apertured with long apertures that form two handles 245 making it possible for the patient 10 to maneuver the mask 200 with ease.

The portable nature of the mask 200 thus allows the patient 10 to adjust the position of this mask 200 manually, in such a way as to be able to place the sighting window 220 in the mask 200 between their dominant eye and the target 120.

Of course, as a variant, provision could be made for the mask 200 to be fixed, in which case the patient 10 would be forced to move their face in order to be able to sight the target 120 through the sighting window 220 in the mask 200.

In a first embodiment of the mask 200, the obstructing panel 210 and the sighting window 220 are passive in the sense that they have light transmission properties that are invariable in time.

In order to allow the obstructing panel 210 to hide the target 120 from at least one of the two eyes 12 of the patient 10, while allowing the camcorder 110 to capture an image of the face of the patient, provision is made for the obstructing panel 210 to have specific optical properties.

Such as shown in FIG. 4, the obstructing panel 210 is formed of a polarizing sheet that polarizes the light in a first sense whereas the sighting window 220 is not polarized (or is optionally polarized perpendicularly or in an opposite sense).

In order to ensure that the obstructing panel 210 of the mask 200 hides the target 120 from at least one of the two eyes of the patient 10, provision is made to equip the latter with a pair of spectacles 400 comprising a frame equipped with two rims 402, and two lenses 401 that are mounted in the rims 402 and that are polarized in a second direction (opposite or perpendicularly to the first direction).

Thus, the patient 110 sees the target 120 only through the sighting window 220 in the mask 200 alone.

The polarization of the obstructing panel 210 and of the lenses 401 may be rectilinear. The obstructing panel 210 could then be vertically polarized whereas the lenses 401 would be horizontally polarized (in the position worn by the patient). The particularity of this type of polarization is that, when the patient 10 inclines their head or the mask, the polarization directions of the lenses 401 and of the obstructing panel 210 are no longer perfectly orthogonal, thereby making the obstructing panel 210 slightly transparent for the patient 10.

The polarization of the obstructing panel 210 and of the lenses 401 may also be circular. The obstructing panel 210 could then be polarized in the clockwise direction whereas the lenses 401 would be polarized in the anticlockwise direction. In this way, the inclination of the head of the patient 10 does not influence the opacity of the obstructing panel 210 seen by the patient 10.

As FIG. 4 shows, in the sighting position the patient 10 can see the target only with just one of their two eyes 12, whereas the camcorder 110 may acquire an image of the face of the patient in which the two lenses 401 of the pair of spectacles 400, and the dominant eye of the patient 10, appear.

As a variant, provision could be made for the pair of spectacles 400 to be replaced by a piece of equipment comprising two polarized lenses, and means for fastening these two polarized lenses to a pair of conventional spectacles with which the patient is equipped.

As another variant, the pair of spectacles 400 could be replaced by a piece of equipment comprising a polarized, for example rectangular, plate, and means for fastening this polarized plate to a conventional pair of spectacles with which the patient is equipped.

As another variant of the mask 200 in which the obstructing panel 210 and the sighting window 220 are passive, provision could be made for the obstructing panel 210 to have a light transmission level at least three times higher in one direction than in the other.

It could even have a nonzero light transmission level in one direction (from the patient toward the camcorder) and a zero light transmission level in the other direction (from the target toward the patient). By way of example, the obstructing panel 210 could thus be formed by a semireflective (or half-silvered) mirror. Thus the camcorder 110 will be able to film the face of the patient 10 while the obstructing panel 110 hides the target 120 from at least one of the two eyes 12 of the patient.

In a second embodiment of the mask 200, the obstructing panel 210 is active in the sense that it has light transmission properties that are variable in time.

In this embodiment, the optical properties of the obstructing panel 210 are controlled between a darkened state, in which the obstructing panel 210 hides the target 120 from at least one of the two eyes 12 of the patient 10, and a transparent state, in which the obstructing panel 210 allows the face of the patient to be seen by the camcorder 110.

Such as shown in FIG. 5, the obstructing panel 210 is formed, with the sighting window 220, by a liquid-crystal screen.

The sighting window 220 is then controlled to remain in the transparent state continuously. In contrast, the opacity of the obstructing panel 210 is controlled to the darkened state while the patient is seeking to sight the target 120, then to the transparent state when the patient has the target 120 in their line of sight. Thus, by acquiring an image of the face of the patient at the exact moment when the obstructing panel 210 passes to the transparent state, a photograph is obtained showing the patient 10 sighting the target 120.

Independently of whether the obstructing panel 210 is active or passive, controlling means are provided, especially for controlling the camcorder 110.

Such as shown in FIG. 1, these controlling means comprise:
a trigger 231 easily accessible by the patient 10 since it is located on the chassis 240 of the mask 200 beside one of the handles 245;
an infrared transceiver 230 located on the chassis 240 of the mask 200, for transmitting a signal relating to whether the trigger 231 is in the depressed or released position;
an infrared transceiver 130 located on the totem 100, for receiving the signal relating to whether the trigger 231 is in the depressed or released position; and
an information processing unit 140 for controlling the camcorder 110 depending on the position of the trigger 231.

If the obstructing panel 210 is of the inactive type, the information processing unit 140 is more precisely adapted to control the camcorder 110 in such a way that the latter acquires an image of the patient 10 as soon as the latter depresses the trigger 231.

If the obstructing panel 210 is of the active type, the information processing unit 140 is adapted to control the obstructing panel 210 so that it passes from the darkened state to the transparent state as soon as the patient 10 depresses the trigger 231, then to control the camcorder 110 in such a way that the latter acquires an image of the patient 10.

Preferably, as will also be described hereinbelow, the information processing unit 140 will moreover also be adapted to deduce from the acquired image which of the two eyes 12 of the patient 120 is their dominant eye.

The determining device 1 shown in FIG. 1 lastly comprises a display screen 300, such as an LCD screen, adapted to display the image of the patient acquired by the camcorder 110. This display screen 300 is then controlled by the information processing unit 140.

The image of the face of the patient displayed on the display screen may moreover optionally have been processed beforehand (for example brightened) and/or additional information, such as indicators specifying the “right-hand” and “left-hand” sides of the image, or specifying which of the two eyes 12 of the patient 10 is their dominant eye, may be superposed thereon.

The method for determining the dominant eye of the patient 10 using the aforementioned determining device 1 is then implemented in the following way, in five main steps, i.e.:
a step in which the patient 10 and the mask 200 are positioned in the field of the camcorder 110, in such a way that the target 120 is hidden from at least one of the two eyes 12 of the patient 10 by the obstructing panel 210 of the mask 200;
a step in which the patient 10 sights the target 120 through the sighting window 220 in the mask 200;
a step in which an image of the face of the patient 10 is acquired by the camcorder 110;
a step in which the dominant eye of the patient 10 is deduced depending on the position of the mask 200 and of the face of the patient 10 in the acquired image; and
a step of displaying the acquired image on the display screen 300.

In the positioning first step, the optician sits the patient 10 on the chair 20 facing the totem 100 and indicates to them the position of the target 120 in order to identify it to them.

The position of the chair 20 may then be adjusted in such a way that, ideally, the face of the patient 10 is located in front of the objective lens of the camcorder 110.

This ideal position may be more precisely defined by characterizing the frame of reference of the face of the patient 10 by means of three planes P₁, P₂, P₃ that lie orthogonal to one another.

Such as shown in FIG. 2, it is thus possible to define a Frankfurt plane P₁ passing through the inferior orbital margins and the porion of the patient (the porion being the highest point in the skull of the ear canal, which corresponds to the tragion of the ear). This Frankfurt plane P₁ must then ideally be positioned substantially horizontally and pass through the optical axis A1 of the camcorder 110.

It is also possible to define a sagittal plane P₂ as being the plane perpendicular to the Frankfurt plane P₁, and that passes through the perpendicular bisector of the two eyes 12 of the patient 10 (the perpendicular bisector being that axis which passes through the middle of the segment defined by the centers of rotation of the two eyes 12, and which lies parallel to the Frankfurt plane P₁). This sagittal plane P₂ must then ideally be positioned vertically and pass through the optical axis A1 of the camcorder 110.

Lastly, a frontal plane P₃ may be defined as being the plane that is perpendicular to the Frankfurt plane P₁ and the sagittal plane P₂ and that passes through the apex of the head of the patient 10. This frontal plane P₃ must then ideally be positioned orthogonally to the optical axis A1 of the camcorder 110.

Of course, the image of the face of the patient 10 may be acquired when the head of the patient is slightly inclined or shifted relative to this ideal position.

However, the image is preferably acquired when the optical axis A1 of the camcorder 110 makes an angle of between −20 and +20 degrees with the frontal plane P₃ of the head of the patient 10, in order to prevent any measurement error.

The optician then asks the patient 10 to take the mask 200 by its handles 245, then to hold it at arm's length in order to interpose it between their face and the target 120. Thus positioned, the mask 200 lies on the optical path of the light emitted by the target 120 toward the patient.

The sighting second step consists, for the patient 10, in moving the mask 200 into a position in which they have the target 120 in their line of sight, through the sighting window 220, while preferably keeping both eyes open.

This step must allow the patient 10 to place the mask 200 naturally in a position such that they can observe the target 120 with their dominant eye.

In practice the patient 10 naturally chooses one or other of their two eyes 12, i.e. their dominant eye, to observe the target 120 through the sighting window 220.

If the patient 10 is unsure, the optician may ask the patient 10 to position the mask 200 near their face and then to slowly move it away therefrom while keeping the target in their line of sight with one, then the other, of their two eyes 12. The patient 10 will generally have difficulty carrying out this operation with one of their two eyes 12 whereas they will find it easy to carry out this operation with the other of their two eyes 12, which in practice is their dominant eye.

The third step of acquiring an image of the patient 10 is here controlled by the patient themselves, when the target 120 is in their line of sight.

Thus, when the patient 10 presses the trigger 231, the information processing unit 140 is programmed to control acquisition of a photograph of the face of the patient (optionally after having controlled the obstructing panel 210 to its transparent state).

As a variant, provision could be made for the optician to have a remote control at their disposal in order for them to control this image acquisition themselves when they consider the patient 10 to be in the correct position.

As another variant, provision could be made for the camcorder 110 to acquire images of the face of the patient 10 continuously or at regular intervals, and for the information processing unit 140 to select one of these images in order to determine from this image which of the two eyes 12 of the patient 10 is their dominant eye.

Of course this image will not be randomly selected.

By way of example, provision could be made for the camcorder 110 to acquire an image of the face of the patient 10 once a second, then for the information processing unit 140 to determine, in each of these images, the position of the face of the patient 10 relative to the mask 200. The information processing unit 140 may then select the fifth consecutive image in which the position of the face of the patient 10 relative to the mask 200 has remained substantially stable. In other words, the image will be selected when the patient 10 has kept the target 120 in their line of sight with the same eye for a preset length of time, here equal to 5 seconds.

In this variant, if the screen is of the active type, provision may especially be made for the obstructing panel 210 to pass from the darkened state to the transparent state regularly (here every second), for a length of time that is sufficiently short that this change of state is imperceptible to the patient, but sufficiently long that the camcorder 110 is able to acquire a clear image of the face of the patient 10.

Independently of the way in which the image is acquired, this image will then preferably contain the (transparent) mask 200 and both eyes 12 of the patient 10 or the two rims 402 of the pair of spectacles 400 worn by the patient 10 (see FIG. 4).

The fourth step then consists in deducing, from this single image, which of the two eyes 12 of the patient 10 is their dominant eye.

A wide range of image processing techniques may be used for this purpose, such as one of the following four techniques.

The first technique is applicable only when both eyes 12 of the patient 10 appear in the acquired image.

This first technique consists in calculating, in the plane of the image, on the one hand, the coordinates of the pupils (or the commissures) of the two eyes 12 of the patient 10, and on the other hand, the coordinates of the center of the sighting window 220 in the mask 200.

The dominant eye is then the eye the coordinates of which are closest to those of the center of the sighting window 220.

The second technique, which is analogous to the first, is applicable only when the two rims 402 of the pair of spectacles 400 worn by the patient 10 (see FIG. 4) appear in the acquired image.

This second technique then consists in calculating, on the one hand, the coordinates of the barycenter of each of the two rims 402 of the pair of spectacles 400, and on the other hand, the coordinates of the center of the sighting window 220 in the mask 200.

The dominant eye is then the eye located behind the rim 402 the coordinates of the barycenter of which are closest to those of the center of the sighting window 220.

The third technique is more flexible since it is applicable whether both eyes of the patient 10 can be seen in the acquired image or not.

This technique will preferably be used when the acquired image is particularly dark and the two eyes 12 of the patient 10 can only be seen with difficulty in this image.

This third technique consists in calculating, on the one hand, the coordinates of the center of the mask 200, by virtue of marks 250 produced on the chassis 240 of the mask 200 (these coordinates corresponding to the coordinates of the center of the sighting window 220), and on the other hand, the coordinates of the barycenter of the outline of the face of the patient 10. Specifically, this outline can be seen more easily than the eyes 12 of the patient 10.

The dominant eye of the patient is then their left eye if the barycenter of the outline of the face of the patient 10 is shifted to the left relative to the center of the mask 200. In contrast, their dominant eye will be their right eye if the barycenter of the outline of the face of the patient 10 is shifted to the right relative to the center of the mask 200.

The fourth technique is applicable when only one of the two eyes 12 of the patient 10 (FIG. 4), namely their dominant eye, appears in the acquired image.

This fourth technique then consists in calculating the shape of the eye of the patient, in observing the left- or right-hand position of the bulbar conjunctiva of the eye, and in deducing therefrom if the dominant eye of the patient 10 is respectively their left eye or their right eye.

The displaying fifth step consists, for the information processing unit 140, in controlling the display of the acquired image on the display screen 300.

Of course, this image may be processed beforehand, for example in order to brighten it.

The information processing unit 140 may also control the display, superposed on the acquired image, of indicators signaling which of the sides of the image is the right-hand side and the left-hand side (which is particularly useful if the displayed image is a mirror image).

The information processing unit 140 may also control the display, beside the acquired image, of a message indicating which of the two eyes 12 of the patient 10 is their dominant eye.

Thus, by virtue of this displaying step it is easy to convince the patient 10 which of their two eyes 12 is their dominant eye.

The present invention is in no way limited to the embodiments described and illustrated and those skilled in the art will be able to envision many variants.

In particular, the step of deducing the dominant eye of the patient could be carried out by the optician or by the patient themselves, and not by an information processing unit.

This deducing step, combined with the displaying step, will then simply consist in the information processing unit 140 displaying, on the one hand, the acquired image on the display screen 300, and, optionally, on the other hand, indicators indicating the left- and right-hand sides of the image. Thus the optician and the patient will easily be able to deduce therefrom which of the two eyes 12 of the patient 10 is their dominant eye.

As another variant of the invention, provision may be made for the outline of the objective lens of the camcorder 110 to be painted red and thus form the target. In this way, when the patient 10 sights the target 120 they will direct their gaze along the optical axis of the camcorder 110.

As another variant of the invention, provision may be made, in the aforementioned positioning, sighting and acquisition steps, for the patient 10 not to be seated but standing.

As another variant of the invention, provision may be made for the target 120 to be located not in the plane of the objective lens of the camcorder 110 but in front or behind this plane, without this position influencing the implementation of the described method.

As one variant of the mask 200, in which the obstructing panel 210 is active, provision may be made for the mask to comprise a transparent sheet covered, except in the sighting window 220, with an optically activatable layer adapted to be controlled between a darkened state and a transparent state.

1. A device for determining the dominant eye of a patient, comprising:
a target visible by the patient in a measuring position;
acquisition means for acquiring an image of the face of the patient in the measuring position; and
a mask to be positioned between, on the one hand, the face of the patient in the measuring position, and on the other hand, the target and the acquisition means, comprising:
a) an obstructing panel:
a1) that has dimensions such that it is capable of being interposed between the target and the two eyes of the patient in the measuring position;
a2) that transmits, at least momentarily, only at most some of the light propagating from the target toward the patient in order to hide the target from the patient in the measuring position; and
a3) that transmits, at least momentarily, at least some of the light propagating from the patient toward the acquisition means in order to allow the acquisition means to acquire an image of the face of the patient in the measuring position; and
b) a sighting window used by the patient to sight the target:
b1) that is located in the obstructing panel;
b2) that transmits the light propagating from the target toward the patient, in order to allow the patient to sight the target; and
b3) that has dimensions such that the target (120) is visible only by a single eye of the patient (10) in the measuring position.

2. The device as claimed in claim 1, in which the obstructing panel and the sighting window are passive in the sense that they have light transmission properties that are invariable in time.

3. The device as claimed in claim 2, in which the obstructing panel polarizes light linearly or circularly, and in which the patient is equipped with a pair of spectacles or equivalent which comprises lenses that are polarized linearly or circularly, respectively, the lenses and the obstructing panel being rectilinearly polarized in orthogonal directions or circularly polarized in opposite directions.

4. The device as claimed in claim 1, in which the obstructing panel is active in the sense that it has variable light transmission properties.

5. The device as claimed in claim 4, in which the obstructing panel and the sighting window together form a liquid-crystal screen.

6. The device as claimed in claim 4, in which the sighting window is inactive in the sense that it has invariable light transmission properties, and in which the obstructing panel comprises an activatable layer controlled to exhibit complete opacity or transparency.

7. The device as claimed in claim 1, in which a display screen is provided, said display screen being suitable for displaying an image of the patient in the measuring position, said image being acquired by the acquisition means.

8. The device as claimed in claim 1, in which an information processing unit is provided, said information processing unit being capable of processing an image of the patient in the measuring position and of deducing therefrom the dominant eye of the patient, said image being acquired by the acquisition means.

9. The device as claimed in claim 1, in which the mask comprises a chassis equipped with at least one handle, and is portable by the patient.

10. A method for determining the dominant eye of a patient using a device comprising a target, acquisition means for acquiring images, and a mask comprising an obstructing panel apertured with a sighting window, comprising:
a step in which the patient and the mask are positioned in the field of the means for acquiring images, in such a way that the mask is located between the target and the two eyes of the patient;
a step in which the patient sights the target through the sighting window;
a step in which the acquisition means acquires an image of the patient sighting the target, in which image at least part of the face of the patient and of the mask appear; and
a step in which the dominant eye of the patient is deduced depending on the position of the mask relative to the face of the patient in the acquired image.

11. The method as claimed in claim 10, in which the deduction step consists in displaying the acquired image on a display screen.

12. The method as claimed in claim 10, in which, in the deduction step, the acquired image is processed in order to calculate the positions of the two eyes of the patient, and the position of the sighting window relative to the two eyes of the patient.

13. The method as claimed in claim 10, in which, the patient being equipped with a pair of spectacles, in the deduction step, the acquired image is processed in order to calculate, on the one hand, the positions of the two rims of the pair of spectacles, and on the other hand, the position of the sighting window relative to the two rims.

14. The method as claimed in claim 10, in which, the mask comprising at least one visible reference mark, the acquired image is processed in order to calculate the position of the face of the patient relative to the visible reference mark.

15. The method as claimed in claim 10, in which the image acquisition is manually controlled.

16. The method as claimed in claim 10, in which the image acquisition is carried out continuously or at regular intervals, and in which, in the deduction step, provision is made for one of said acquired images to be selected.

17. The method as claimed in claim 14, in which the image is selected when the position of the face of the patient relative to the mask is stabilized.

18. The method as claimed in claim 10, in which a step of displaying the acquired image is provided.
